(19) United States
(12) Patent Application Publication
(10) Pub. No.: US 2013/0310652 A1
(43) Pub. Date: Nov. 21, 2013

(54) INTEGRATED SURGICAL TASK LIGHTING

(71) Applicants: Wael K. Barsoum, Bay Village, OH (US); David P. Gurd, Chagrin Falls, OH (US); John D'Angelo, Twinsburg, OH (US); Douglas R. Johnston, Shaker Heights, OH (US)

(72) Inventors: Wael K. Barsoum, Bay Village, OH (US); David P. Gurd, Chagrin Falls, OH (US); John D'Angelo, Twinsburg, OH (US); Douglas R. Johnston, Shaker Heights, OH (US)

(73) Assignee: THE CLEVELAND CLINIC FOUNDATION, Cleveland, OH (US)

(21) Appl. No.: 13/893,378

(22) Filed: May 14, 2013

Related U.S. Application Data
(60) Provisional application No. 61/647,097, filed on May 15, 2012.

(51) Int. Cl. A61B 19/00

(52) U.S. Cl. CPC A61B 19/5202; A61B 19/5225
USPC 600/249

(57) ABSTRACT

Method and apparatus for coordinated control of lights and devices in an operating room. An operating room is equipped with a plurality of devices, each capable of generating light. A set of rules are provided defining the manner in which the plurality of devices are to be controlled in a cooperative, coordinated manner in accordance with the state of the operating room. The state of the operating room is detected, as such state changes from time to time during the course of a medical procedure being performed in the operating room. The plurality of devices are controlled in accordance with the detected state, as it changes from time to time, and the set of rules. The plurality of devices are thus controlled in a cooperative, coordinated manner in accordance with the changing state of the operating room.

### RELATED APPLICATION

This application claims priority from U.S. Provisional Patent Application Ser. No. 61/647,097, filed 15 May 2012, the subject matter of which is incorporated hereby incorporated by reference in its entirety.

### FIELD OF THE INVENTION

The present invention is directed to methods and apparatus for lighting surgical operating rooms.

### BACKGROUND

Surgical procedures are performed in large, sophisticated operating rooms. The patient undergoing the procedure is positioned on an operating table near the middle of the room. The operating table is of course the center of all activity in the room and is surrounded by patient monitoring systems, lighting systems, and medical tools and support services. The surgical team must also be deployed around the operating table, and there is a natural contention for space. Stringent protocols must be observed for maintaining sterility within the operating theater. Moreover, since time is always of the essence in a surgical procedure, all actions and processes—including those associated with control of operating room systems—must be performed in as smooth and efficient a manner as possible. In particular, the lead surgeon must have the ability to control the various systems, preferably without actually touching the devices and systems and without undue complexity. Each such system must perform its respective function effectively while not interfering with the surgeon's ability to focus his concentration on the surgical procedure under way.

Since surgery is, at root, a manual procedure, the surgeon must be able to see clearly the region of surgical interest. Bright surgical lights are therefore employed to provide uniform high levels of illumination. In fact, there are many sources of illumination in the operating room, including not only the surgical lighting, but also task lighting, headlamp lighting, brightly lit electronic monitors, and ambient lighting. All of these illumination sources compete for visual attention and require individual control. Moreover, they all take space, generate heat, and include many surfaces, corners, seams, and edges that can render regular sterilization arduous and time consuming.

### SUMMARY OF THE INVENTION

The present invention provides methods and apparatus for convenient, coordinated control of illumination sources within in operating room.

In accordance with one example embodiment of the present invention, a method is provided for coordinated control of lights and devices in an operating room. The operating room is equipped with a plurality of devices, each capable of generating light for illuminating part or all of the operating room or for providing perceptible displays of information, each such device being individually controllable to increase or decrease the amount of light generated thereby. A set of rules is provided defining the manner in which the plurality of devices are to be controlled in a cooperative, coordinated manner in accordance with the state of the operating room. The state of the operating room is detected, as such state changes from time to time during the course of a medical procedure being performed in the operating room. The plurality of devices is controlled in accordance with the detected state, as such state changes from time to time, and the set of rules. The plurality of devices is thus controlled in a cooperative, coordinated manner in accordance with the changing state of the operating room.

In accordance with another example embodiment of the present invention, a method is provided for illuminating a region of surgical interest in an operating room. The room is equipped with an array of overhead spotlights, each spotlight being selectively controllable to brighten or dim the amount of illumination provided thereby. Each of the spotlights is oriented and focused so as to illuminate only a preset portion of the region of surgical interest. The portion of the region of surgical interest to be illuminated is determined. The spotlights are controlled such that those of the spotlights that are focused on the determined portion of the region of surgical interest are illuminated more brightly than other spotlights in the array of overhead spotlights.

In accordance with yet another example embodiment of the present invention, apparatus is provided for illuminating a region of surgical interest in an operating room. The apparatus includes an array of spotlights fixed to the ceiling of the operating room, each spotlight being selectively controllable to brighten or dim the amount of illumination provided thereby. Pointing and focusing elements orient and focus each spotlight on an associated preset portion of the region of surgical interest. A control circuit is provided for controlling the array of spotlights. The circuit includes at least one input device for determining which portion of the region of surgical interest that is to be illuminated, and a light controller for the array of spotlights. The light controller selectively energizes the spotlights of the array of spotlights in response to the determined portion such that those of the spotlights focused on the determined portion of the region of surgical interest are illuminated more brightly than other spotlights in the array of overhead spotlights.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIGS. 1 and 2 are simplified perspective views of an operating room In accordance with one example embodiment of the present invention;

FIGS. 3 and 4 are simplified perspective views of the manner in which the overhead lamps of the operating room of FIGS. 1 and 2 are focused on individual portions of the area of surgical interest;

FIG. 5 is a block diagram of the electronic system used to provide coordinated control of the illumination sources in the operating room of FIGS. 1 and 2; and,

FIG. 6 is a simplified flow chart of the process performed by the electronic system of FIG. 5.

### DETAILED DESCRIPTION

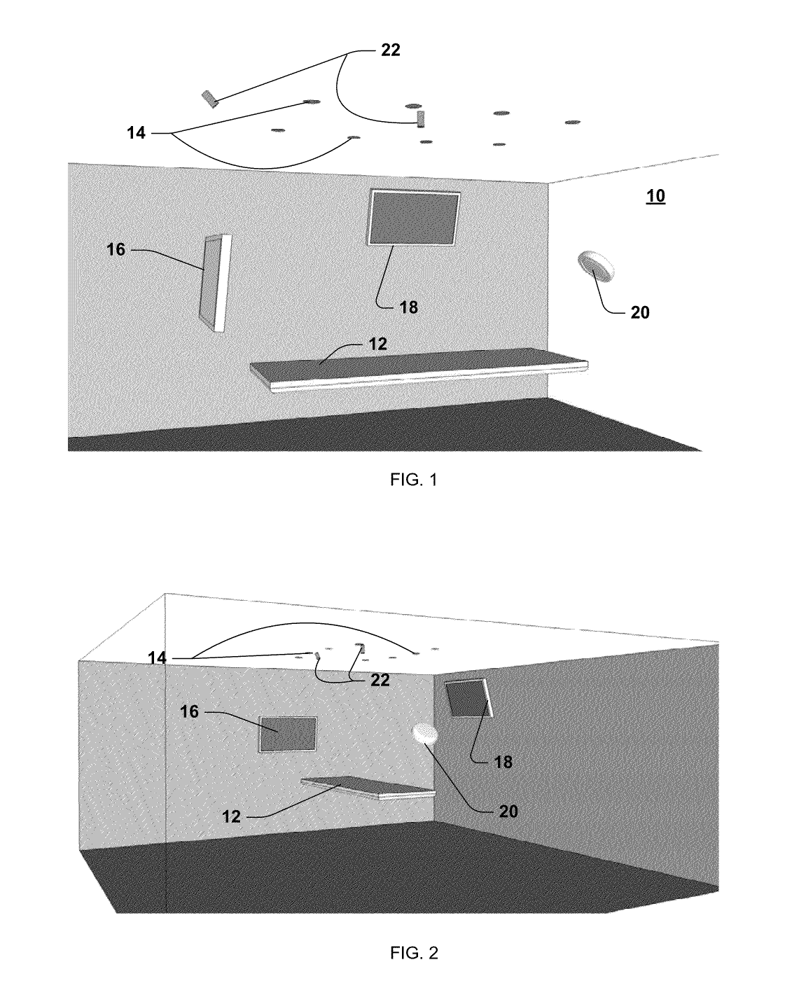

Referring to FIGS. 1 and 2, one example embodiment is shown of an operating room 10 in accordance with the present invention. Operating rooms are typically equipped with a substantial number of sophisticated support systems and medical devices. For simplicity of illustration and convenience of description, most of these systems have been omitted from FIGS. 1 and 2. For those pieces of operating room equipment that have been shown, conventional brackets, pedestals, and supports have been omitted for the same reason.

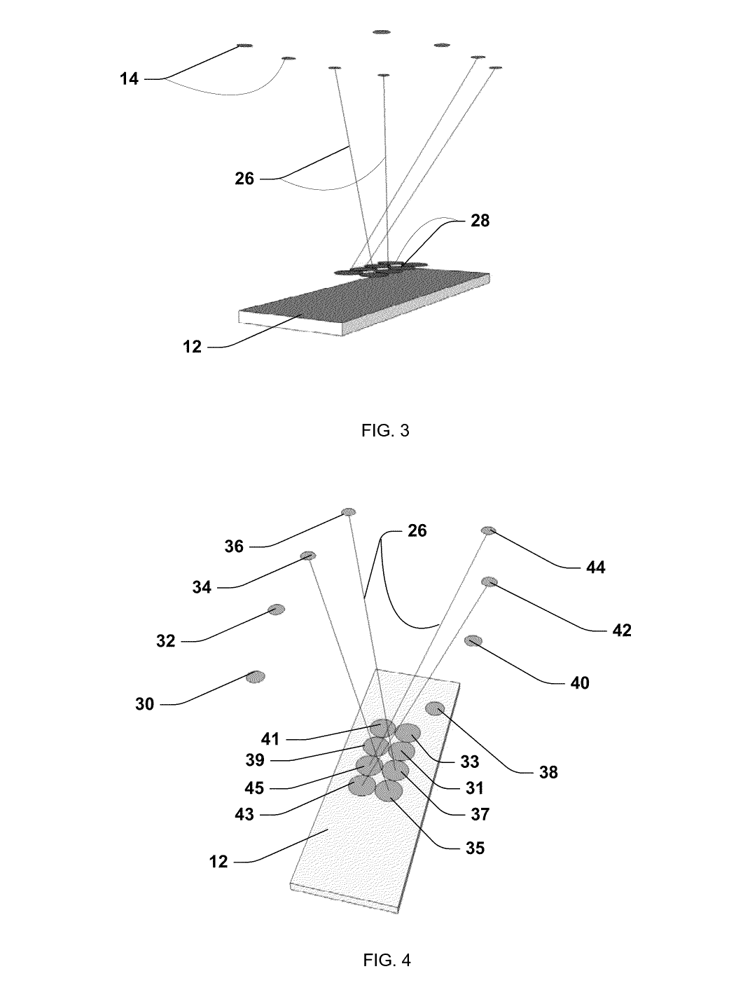

The operating room 10 as shown in FIG. 1 has an operating table 12 located approximately in the middle of the room. Surgical lighting, which conventionally is provided by one or more rather massive light fixtures mounted on articulated arms above the operating table, is instead provided by an array of overhead spotlights 14 embedded in the operating room ceiling. These will be discussed further in connection with FIGS. 3 and 4.

There are a number of other illumination sources within the room, including ambient lighting (not separately shown, but preferably installed in the ceiling or walls of the operating room 10), touch sensitive LCD or LED panels 16 for controlling various operating room systems, and display monitors 18 (also LCD or LED) for displaying images, or text and other data provided by operating room systems. In addition, task lighting 20 is provided to augment the overhead lighting as required, and headlamps (not shown in FIGS. 1 and 2) are provided for use by the lead surgeon and/or other assisting surgeons. In accordance with the present invention, these sources of illumination are controlled in a coordinated, integrated manner without requiring the surgeons to touch the various devices.

Video and audio circuits are included to assist in the integrated control of the room illumination. One or more video cameras 22 is installed in the ceiling of the operating room 10 and the surgeon headlamps include microphones for listening to voice commands issued by the surgeon. The cameras and microphones may, in addition to serving the control purpose, also provide passive room monitoring that may be recorded for the benefit of training, for example.

FIGS. 3 and 4 are simplified perspective representations of the overhead array 14 of spotlights and their orientation with respect to the operating table 12. The figures show the optical axes 26 of several of the spotlights and the respective illuminated portions 28 of the region of surgical interest. The phrase “region of surgical interest,” as used herein, refers to the part of the patient upon which the surgical procedure is to be performed. The patient will be lying on the operating table 12.

In the figures, the array 14 includes eight individual spotlights. However, it is anticipated that the array will include a larger number of spotlights, possibly several dozen or more. The individual spotlights are generally similar to one another. Each spotlight includes multiple light-emitting diodes (“LED”) for generating bright illumination of the proper color temperature without generating significant excess heat. The LED's are dimmable so that the amount of light generated by each spotlight may be modulated in a fashion to be described hereafter. The illumination thus provided by the LEDs is collimated and focused by lenses and other optical elements such that the illumination from each spotlight is a narrow beam that shines primarily onto a respective small spot or portion 28 of the region of surgical interest. Each spotlight is fixed in place and thus the illuminated portion 28 associated with that spotlight is similarly fixed.

The light beams provided by the respective spotlights are directed to different portions of the region of surgical interest, much like the squares of a checkerboard, whereby any portion or groups of portions of the region of surgical interest can be illuminated by selective activation of respective ones of the spotlights of the array 14. This can perhaps better be seen in FIG. 4, wherein spotlights 30, 32, 34, 36, 38, 40, 42, and 44 respectively illuminate portions 31, 33, 35, 37, 39, 41, 43, and 45. The optical axes 26 of some, but not all, of the spotlights are shown in FIG. 4. To avoid shadows, preferably the array will include enough spotlights (more than the eight shown in the figures) so that each portion of the region of surgical interest will be illuminated by more than one spotlight. Moreover, the spotlights will preferably each cross the medial plane of the area of surgical interest, whereby the lights will be more likely to shine under overhanging tissue, forceps and the like. In other words, the spotlight will itself be located on the opposite side of the medial plane from the portion that is actually illuminated by that spotlight.

Thus, for example, when portion 37 of the region of surgical interest is the focus of the lead surgeon's attention, spotlight 36 will be brightened to brightly illuminate that portion. The lighting of adjacent portions (e.g. portions 31, 35, and 45) may be dimmed by reducing illumination from the corresponding spotlights. The manner in which this control is achieved will be discussed hereafter.

There will necessarily be some overlap between the portions 28 illuminated by adjacent spotlights, and this effect may be exploited to provide good illumination of even the gaps between the portions 28. Thus, when the surgeon's attention is directed to some area that does not align precisely with one of the portions 28, that area may be illuminated by brightening all adjacent spotlights to an intermediate degree. The area of overlap, which will be illuminated with light contributed from all of the overlapping portions of the beams, will then be more brightly lit than the portions 28 themselves. Thus, any desired portion of the region of surgical interest may be highlighted by selective operation of the spotlights in the array 14.

The beams of the spotlights may be pointed in their respective directions in any convenient manner. For example, the spotlights may all be pointed vertically downward, but may have wedge prisms inserted into each optical path, each wedge prism being of the proper optical geometry to redirect the beam from the vertical towards the proper direction. Alternately, each spotlight simply may be mounted at a corresponding angle to the ceiling by tailored fixtures or brackets. In either case, the mounting structures or optical beam steering elements are conveniently computer designed in accordance with the geometry of the operating room 10, of the array 14, and of the portions 28 of the region of surgical interest, whereby each spotlight will automatically be aligned properly when the spotlight is plugged into its respective location in the ceiling.

Each spotlight has a cover that is hermetically sealed to the ceiling of the operating room, thereby to simplify the establishment and maintenance of sterility of the component and the room in general. The area above the ceiling is devoted to the cans for the spotlights, as well as ambient lighting, cameras and other mechanicals. Fans, not shown, will be provided to give this above-ceiling area a negative atmospheric pressure further to reduce the risk of air infiltration into the operating room.

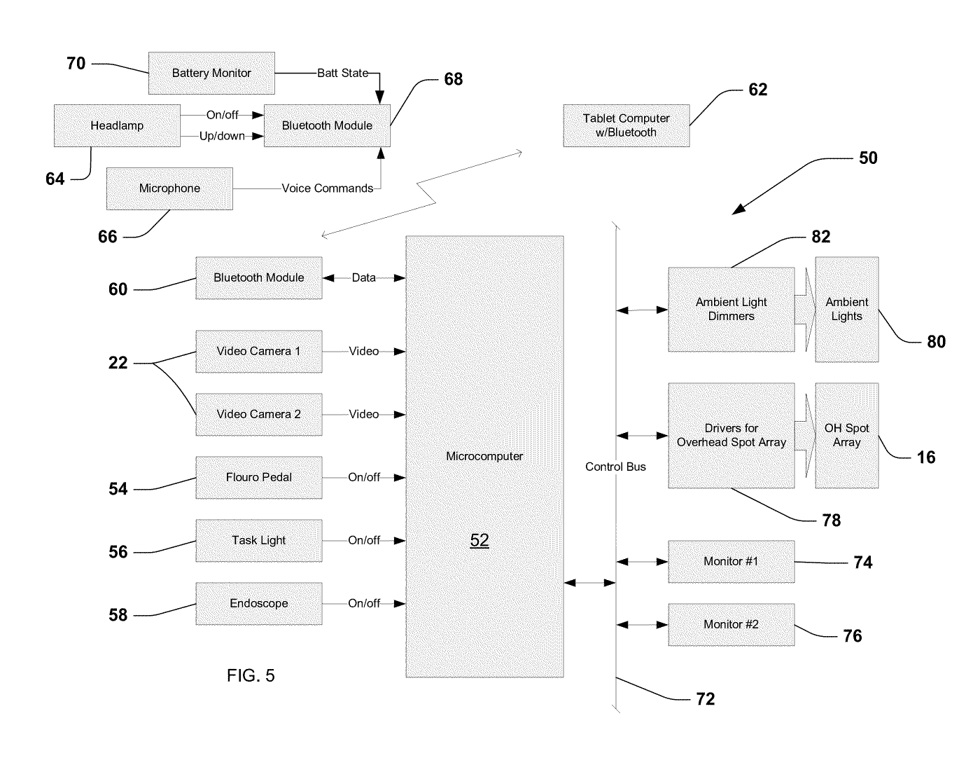

The overhead spotlight array 14 and other sources of illumination in the operating room 10 are controlled in a coordinated, integrated fashion by a lighting control module 50, the content of which is shown in block diagram form in FIG. 5. Microcomputer 52 is of generally conventional design, including a microprocessor, random access and read only memory, input and output interfaces, signal convertors (e.g., analog to digital convertors and digital to analog converters) and other peripheral components. The microcomputer is programmed with operating software designed to control the sources of illumination within the operating room. More specifically the microcomputer is programmed with a hierarchical series of lighting rules defining the manner in which the sources of illumination within the operating room 10 are to work cooperatively together in accordance with the changing state of the operating room, as it changes from time to time during the course of a surgical procedure.

The state the operating room is determined by the actions of the lead surgeon, the assisting surgeon, and other surgical team members during the course of the surgical procedure. When a member of the surgical team manually turns on or off a system that is connected to microcomputer 52, the microcomputer senses that action and adjusts the lighting in accordance with the pre-programmed hierarchal lighting rules. The lead surgeon may also issue commands directly to the lighting module, either through voice commands and/or through hand or body gestures made within the field of vision of the video cameras 22.

To this end, microcomputer 52 receives digital and/or analog signals from multiple systems around the operating room. Video cameras 22 feed analog or digital video signals into microcomputer 52, where pattern recognition software processes the signals to distinguish recognizable command gestures made within the field of view of the camera. A fluoroscope control pedal 54, task light 56, and endoscope 58 will each provide respective digital status signals to microcomputer 52, whereby the status of those devices may be included in the rules-based integrated lighting control process.

Control signals are received wirelessly from other operating room devices via a Bluetooth transceiver module 60. (Although a Bluetooth wireless link is shown, other wireless communications protocols may instead by used, including for example any conventional wifi protocol.) The transceiver 60 communicates directly with one or multiple tablet computers 62, such as Apple Corporation iPad tablets or equivalent devices.

The surgical team will be wearing headsets including the previously mentioned headlamps. The headsets may be hard-wired into the microcomputer by simple cabling but, in the illustrated embodiment, each headset includes not only a headlamp 64 and a microphone 66, but also a Bluetooth transceiver 68 for transmitting to microcomputer 52 both the state of the headlamp and also a digitized audio feed from the microphone. (Microcomputer 52 includes an ‘audio command recognition’ software module that will extract spoken commands from the audio feed.) The headset is battery operated and includes two or more batteries carried in holsters worn on the back of the surgeon's belt for easy change-out by support staff. The headset battery monitor 70 detects a loss of charge on one battery and automatically switches to the alternate battery, while also initiating a red warning lamp on the battery to mark it for replacement and sending a notice via the Bluetooth link for forwarding to one or more of the display panels. The headlamp also includes an accelerometer for detecting the position of the headlamp. At a minimum, the headlamp will use the accelerometer output to generate a position status signal indicating whether the headlamp is pointed down or up. This status signal is sent to microcomputer 52 over the Bluetooth link, along with the other headset signals.

Microcomputer 52 uses the aforementioned status and control signals to access a hierarchal set of lighting rules for the operating room 10. Microcomputer 52 then controls many, most, or all of the sources of illumination within the operating room 10 based on the outcome of the rules. Microcomputer 52 sends appropriate illumination commands to many systems via hard-wired links established with a command and control bus 72. Control bus 72 may comprise any convenient control bus, either a simplified special purpose hard cable or a more general solution (e.g. Ethernet or other local area network protocol). In any case, monitors and touch panels (e.g. monitors 74, 76) will receive brightness instructions from microcomputer 52 and will brighten or dim their respective display, as commanded, or even extinguish the display panel entirely.

The overhead spot array 16 will be controlled by a sub-module power driver 78. The power driver module 78 includes buffers and input/output ports for receiving specific brightness instructions from microcomputer 52 for each spotlight in the array. The instructions are stored within the buffer in non-volatile memory so that a brief power interruption will not result in loss of overhead lighting, and the buffer is updated each time new instructions are communicated from microcomputer 52. The power driver module 78 includes a set of low-voltage power drivers, with one for each LED spotlight. Each low-voltage power driver modulates the power into the corresponding spotlight according to the instruction stored in the buffer memory for that particular spotlight.

Ambient lights 80, which will generally be operating at line voltage (120 VAC, typically, in the United States) rather than low voltage, will be controlled by a sub-module 82, providing dimming and on/off control of the various ambient lights used in the room.

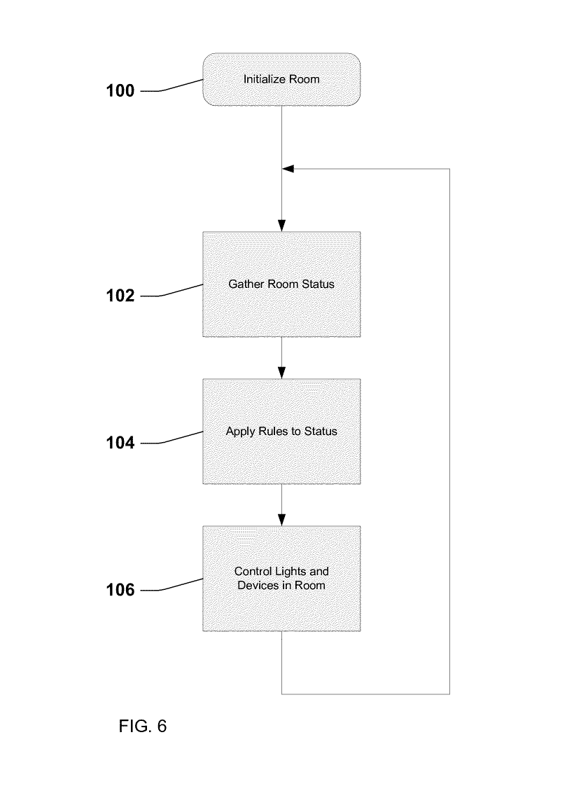

FIG. 6 is a simplified graphic representation of the operating process performed cyclically by microcomputer 52 under software control. When lighting control module 52 is first turned on, it will perform an initialization process at step 100 whereby all controlled systems are put into well defined initial states, flags and buffers are reset, diagnostics are run, etc. Microcomputer 52 will thereafter enter the main processing loop, shown as comprising steps 102-106. In step 102, microcomputer 52 will collect data from all operating room systems as to their present status, thereby determining the present state of the operating room 10. In step 104, the microcomputer will use this system state data to access the stored hierarchal series of lighting rules. Microcomputer 52 will determine which rule or rules apply, according to the defined hierarchy and the existing operating room state. Then, in step 106, microcomputer 52 will send commands to the various controlled sources of illumination within the operating room 10, whereby the controlled lights are brightened and dimmed in an orchestrated, coordinated manner to achieve the lighting effects desired for the existing operating room state.

The rules stored in the memory of microcomputer 52 may change from time to time and room to room depending upon the needs and desires of the lead surgeon and the exigencies of the operating procedure being performed. Different sets of rules may be preset in a single lighting control module, and called up and used for specific operating procedures. Some sets of rules may be preferred by one surgeon or another. Preferably the rules are easily editable in natural language so that they may be changed readily and without uncertainty as to meaning and effect. The rules may, for example, be edited in natural language and standard text on any computer, and then checked for syntax and consistency before being loaded into the lighting control module 50. Some rules will now be described, however it will be understood that the rules will change to suit the needs of the situation. Nonetheless, in each case the benefits of unified, coordinated control will be achieved.

The rules may be positive or negative (proscriptive) in nature. For example, it is contemplated that the room ambient lights will be controlled by the lighting control module 50, and that the manual wall controls will be disabled for the duration of the operating procedure, instead being directly controlled by module 50 in accordance with the set of lighting rules.

As stated previously, the various states of the operating room will be determined by, and activated and deactivated by, the actions of the lead surgeon, assisting surgeon, and other members of the surgical staff. The lead surgeon will give voice commands in a specific format, and the command recognition software module of the light control module 50 will recognize the command and perform the instruction in accordance with the rules. If the surgeon, for example, says “command, spots, on”, microcomputer 52 will turn on the spotlights of the overhead array 16 so that a default set of the lights are turn on at medium brightness. Microcomputer 52 will continue waiting, for another preset interval (e.g. 15 seconds) or until the surgeon speaks a ‘stop’ command, for more verbal commands for the spotlights. If the surgeon continues with the command “position”, microcomputer will monitor the overhead video camera 22, looking for a specific hand gesture in the camera's field of view. Preferably the gesture will be a horizontal hand, palm down, fingers spread. Microcomputer 52 will recognize that distinctive image and will control the lights to move the illuminated spot towards the hand.

The field of view of the overhead camera is fixed relative to the array of overhead spotlights 16. The microcomputer will have stored in memory a correlation between camera grid locations and corresponding light spot portions 28 (FIG. 3). Upon recognizing the grid location of the hand image, microcomputer 52 will command the spot array 16, via the drivers 78, so that the existing highlighted spot of light moves towards the surgeon's hand. Although the spotlights are each fixed, the spot will seem to move in a regular, linear fashion as multiple ones of the overhead spotlights along the desired light path are sequentially turned on, brightened, and then dimmed and turned off. Movement of the spot will appear to be quick but not instantaneous. Microcomputer 52 will continue to track the surgeon's hand until the hand is closed into a first or is abruptly removed from the scene. In this manner, the surgeon can rapidly and conveniently reposition the overhead lights without mechanical movement of any light and without the surgeon actually touching anything.

Alternatively, the hand gesture could be the opposite of the one described above, in order to simulate the ‘grabbing’ of the existing spot of light. That is, the surgeon's hand could be placed in the center of the existing illuminated area and then closed into a fist, thereby simulating grabbing of the light beam. The gesture (open hand placed in beam, then closed into a first) would be observed by the overhead camera and recognized by the microcomputer 52 as meaning that the illuminated area is to move with the movement of the hand. As the closed first is moved, the microcomputer would cause the individual spotlights of the array of lights to be illuminated or extinguished so that the illuminated area would dynamically follow movement of the hand. When the hand would then be opened and fingers spread, the microcomputer 52 would recognize the gesture as meaning that the surgeon had ‘released’ the beam of light and repositioning of the illuminated area is finished. Natural gestures involving two hands could in similar fashion be used to cause the microcomputer 52 to narrow or broaden the illuminated area.

In many cases the surgeon may wish to reposition the lights without diverting his hands from their present task. In such a case, the lights will be directed to a particular position by verbal commands identifying the desired location. The region of surgical interest will be divided into, e.g., a four by three grid of twelve squares, and microcomputer 52 will have preprogrammed instructions operable to cause the spot array 16 to illuminate a selected one or more of the grid squares. The grid thus defined may be the same as or different than the grid described previously with respect to FIGS. 3 and 4. Each grid square will have an associated identification name or number, which could be as simple as ‘grid 8’ for a particular grid location. The surgeon will instruct microcomputer 52 to illuminate the desired area by giving the command ‘position’ followed by the desired grid or group, such as “grid 4” or “row 2” or “quadrant 1”. Microcomputer 52 will recognize the voice command and energize the individual spotlights of the spot array to achieve the commanded spot lighting. Alternately, or in addition, specific sets of sequential spotlight positions could be preloaded into microcomputer 52, with sequence selection and stepping through the selected sequence occurring on voice command by the surgeon.

Moreover, other light spot repositioning approaches are also within the scope of the present invention. It is contemplated, for example, that a patient drape used during the surgery could be provided with a defined opening that is recognizable in the video image captured by video cameras 22, whereby the pattern recognition software embedded in microcomputer 52 will be able to recognize the opening. To facilitate the recognition of the opening, the drape may have a visually distinctive aspect such as for example a particular spectral characteristic (e.g. visual color, reflectivity in IR, etc.) or geometric surface design (e.g. cross-hatched lines). Upon recognizing the drape in the image, the microcontroller 52 will operate the overhead spot array such that the light is moved to a default position at the center of the opening. It may then be moved from this default position by the surgeon via the processes already discussed. The drape could be detected non-visually, instead. E.g. magnetic or ultrasonic sensing techniques could be used for drape sensing, provided that the sensors and their operation do not interfere with the proper operation of other sensitive equipment in the room.

The reverse of this targeting technique could also be used. That is, instead of positioning the spot of light at a gap or opening in a visually recognizable drape, a visually recognizable target could be employed, with the microcomputer 52 identifying the target in the field of view of the video signal and then commanding the overhead spot array 16 via drivers 78 to illuminate the location of the target. The target could be a relatively small disk or other geometric shape having specific optical characteristics (e.g., color, reflectivity, etc.). As long as the target was in the field of view, the light spot would follow the target. The target would be covered, or turned over, or removed from the region of surgical interest in order to fix the lights in the last target position.

The positioning of the lighting provided by the overhead spot array 16 may also be controlled by tablet computers 62, operated by a member of the surgical team or supporting staff. The tablet computers may display a static or dynamic image taken from the video cameras 22, and a touch of a finger on the screen of the tablet could be communicated to microcomputer 52 via Bluetooth module 60 and translated by microcomputer 52 into positioning of the light on the area in the region of surgical interest corresponding to the part of the image that was touched.

Headlamps can be very helpful at providing auxiliary lighting to the surgeon at a point of interest. Unfortunately, the headlamp needs to be turned on and off and, if not turned off when the surgeon raises his head to view a colleague or monitor or other device, the light from the headlamp can temporarily blind or at least discomfort other members of the surgical team. It is contemplated that the headlamp worn by a surgeon will be controlled by voice commands made by that surgeon (“command, headlamp, on”, “command, headlamp, off”) but that also there is a second headlamp control rule whereby the headlamp is temporarily turned off or at least significantly dimmed each time the headlamp state signal indicates that the surgeon has raised his or her head. The headlamp will be returned to full brightness when the surgeon again lowers his/her head. Moreover, as one possible corollary of the same headlamp rule set, the rule may require that the overhead spotlights be dimmed to a preset degree when the headlamp is commanded on, and returned to normal brightness when the headlamp is commanded off.

Other rules may deal with the video displays, regardless of whether the display is part of a touch panels or as a non-touch sensitive data display. When the surgeon's head is down, indicating concentration on the procedure under way, the display panel brightness rule will require that the display panel brightness decline along some slow or fast curve, returning immediately to full brightness when the surgeon raises his head. Of course, if the vision system (or some other included head detection system) is capable of determining the pointing direction of the lead surgeon's head, then the brightening effect may be restricted to the display most nearly associated with the surgeon's head direction. Displays outside of the sterile zone, used by other surgical team members for monitoring patient status, would be excluded from the operation of this rule.

If a fluoroscope is employed in support of the procedure, the activation of the fluoroscope (e.g. by pedal 54 of FIG. 5) can trigger a specific fluoroscope rule. It is contemplated that other lighting sources will be dimmed and will remain dimmed as long as the fluoroscope pedal is depressed, returning to previous brightness thereafter.

Similarly, when an arthroscope/endoscope is in use (e.g., as indicated at 58 in FIG. 5) a endoscope rule can be triggered whereby other lighting sources will be dimmed as long as the scope is in use, returning to previous brightness thereafter. A similar rule will be apply to the use of task lighting, and yet another to the use of an x-ray light box. In each case, the rule will be designed to reduce unnecessary peripheral light sources which might distract from attention on the system or area that is the focus of the moment during the procedure.

As stated previously, the rules will be arranged in a hierarchy so that rules having greater urgency will always take precedence over rules having lower urgency.

A system, including apparatus and related methods, has thus been described that frees the operating room of manual controls, provides simple, efficient, unified, coordinated control of a set of operating room light sources, and secures a preferred lighting arrangement under multiple different operating room situations.

From the above description of the invention, those skilled in the art will perceive improvements, changes and modifications. Such improvements, changes and modifications within the skill of the art are intended to be covered by the appended claims.

Having described the invention, the following is claimed:

1. A method for coordinated control of lights and devices in an operating room comprising the steps of:
equipping an operating room with a plurality of devices, each capable of generating light for illuminating part or all of said operating room or for providing perceptible displays of information, each such device being individually controllable to increase or decrease the amount of light generated thereby;
providing a set of rules defining the manner in which said plurality of devices are to be controlled in a cooperative, coordinated manner in accordance with the state of the operating room;
detecting the state of the operating room, as such state changes from time to time during the course of a medical procedure being performed in the operating room; and
controlling said plurality of devices in accordance with said detected state, as such state changes from time to time, and said set of rules,
whereby said plurality of devices are controlled in a cooperative, coordinated manner in accordance with the changing state of the operating room.

2. A method as set forth in claim 1, wherein said operating room includes an operating table having a surface defining a region of surgical interest, and wherein said equipping step includes the step of equipping said room with an array of controllable overhead spotlights, and orienting and focusing each said spotlight on an associated preset portion of said region of surgical interest, whereby any portion of said region can be selectively illuminated by controlling individual spotlights in said array of overhead spotlights.

3. A method as set forth in claim 1, wherein said equipping step includes the step of equipping said room with an array of controllable overhead spotlights illuminating selected regions of said operating room, and wherein said step of controlling comprises the steps of selecting particular spotlights according to the areas illuminated by said particular spotlights and said detected state of said operating room, and illuminating said selected ones of spotlights in said array.

4. A method as set forth in claim 3, wherein said detecting step includes the step of monitoring the actions of at least one person in said operating room.

5. A method as set forth in claim 4, wherein said monitoring step includes the step of detecting gestures made by said at least one person and interpreting said gestures as lighting commands.

6. A method as set forth in claim 5, wherein said operating room includes an operating table having a surface defining a region of surgical interest and wherein said step of interpreting comprises the step of determining from said gestures which part of said region of surgical interest is to be illuminated.

7. A method as set forth in claim 6, wherein said step of selecting particular spotlights comprises the step of selecting spotlights illuminating said determined part of said region of surgical interest.

8. A method as set forth in claim 4, wherein said step of monitoring the actions of at least one person in said operating room comprises the step of monitoring at least one of verbal command or physical gestures.

9. A method as set forth in claim 8, wherein said step of monitoring comprises the step of monitoring hand gestures made by at least one person.

10. A method as set forth in claim 9, wherein said step of monitoring hand gestures comprises the step of monitoring the position of said hand over said region of surgical interest and wherein said step of selecting particular spotlights comprises the step of selecting spotlights illuminating the position of said hand whereby, at least some of the time, the illumination appears to follow the changing position of said hand.

11. A method as set forth in claim 1, wherein at least one of said set of rules comprises a rule governing the manner in which general room lights are to be adjusted in accordance with the state of the operating room.

12. A method as set forth in claim 1, wherein said step of providing a set of rules comprises the step of providing rules governing light generating devices to take effect upon activation of at least one of a fluoroscope, arthroscope, or endoscope.

13. A method as set forth in claim 12, wherein said step of detecting the state of the operating room comprises the step of detecting the state of foot pedal associated with a fluoroscope situated in said operating room.

14. A method as set forth in claim 1, wherein said step of detecting the state of the operating room includes the step of detecting to where the attention of an individual in said operating room is directed.

15. A method as set forth in claim 14, wherein said step of detecting to where the attention of an individual in said operating room is directed comprises the step of detecting at least one of the position or orientation of at least one of the head, eyes, and hands of said individual.

16. A method as set forth in claim 14, wherein said step of providing a set of rules defining the manner in which said plurality of devices are to be controlled comprises the step of providing at least one rule defining the way in which the illumination of a first device is to be adjusted when the attention of an individual in said operating room is directed to said first device.

17. A method as set forth in claim 16, wherein said step of providing said set of rules defining the manner in which said plurality of devices are to be controlled further includes the step of providing at least one rule defining the way in which the illumination of a second device is to be adjusted when the attention of an individual in said operating room is directed to said first device.

18. A method as set forth in claim 14, wherein said step of providing a set of rules defining the manner in which said plurality of devices are to be controlled comprises the step of providing a rule specifying that the illumination of at least one device is to be increased when the attention of an individual in said operating room is directed to said one device while also specifying that the illumination of another device is to be decreased.

19. A method as set forth in claim 1, wherein said step of detecting the state of the operating room comprises the step of detecting the orientation of a device worn by an individual in said operating room.

20. A method as set forth in claim 1, wherein said step of detecting the state of the operating room comprises the step of detecting the state of a foot-actuated switch.

21. A method as set forth in claim 1, wherein said step of detecting the state of the operating room comprises the step of detecting room state commands entered into a data entry device by an individual in said operating room.

22. A method of illuminating a region of surgical interest in an operating room, comprising the steps of:
equipping the room with an array of overhead spotlights, each said spotlight being selectively controllable to brighten or dim the amount of illumination provided thereby,
orienting and focusing each said spotlight so as to illuminate only a associated preset portion of said region of surgical interest,
determining which portion of said region of surgical interest is to be illuminated, and
controlling the spotlights such that those of said spotlights focused on said determined portion of said region of surgical interest are illuminated more brightly than other spotlights in said array of overhead spotlights.

23. A method a set forth in claim 22, wherein said step of controlling said spotlights comprises the step of turning on spotlights that illuminate said determined portion of said region of surgical interest and turning off at least some of the spotlights that do not illuminate said determined portion of said region of surgical interest.

24. A method as set forth in claim 22, wherein said step of controlling said spotlights further comprises the step of automatically causing said spotlights to turn on and off so that, as said determined portion of said region of surgical interest changes, such determined portion remains illuminated while other regions are not.

25. A method as set forth in claim 22, wherein said step of determining which portion of said region of surgical interest is to be illuminated comprises the step of imaging at least said region of surgical interest and processing the resulting image thereby to detect the portion of said region of surgical interest that is to be illuminated.

26. A method as set forth in claim 25, wherein said step of processing the resulting image comprises the steps of detecting gestures of individuals within said image and selecting said portion of said region of surgical interest from said detected gestures.

27. A method as set forth in claim 22, wherein said step of determining which portion of said region of surgical interest is to be illuminated comprises the step of manually identifying said portion on a flat surface.

28. Apparatus for illuminating a region of surgical interest in an operating room, comprising:
an array of spotlights fixed to the ceiling of said operating room, each said spotlight being selectively controllable to brighten or dim the amount of illumination provided thereby,
pointing and focusing elements for orienting and focusing each said spotlight on an associated preset portion of said region of surgical interest,
a control circuit for controlling the array of spotlights, said circuit including at least one input device for determining which portion of said region of surgical interest is to be illuminated, and a light controller for the array of spotlights, said light controller selectively energizing said spotlights of said array of spotlights in response to the determined portion such that those of said spotlights focused on said determined portion of said region of surgical interest are illuminated more brightly than other spotlights in said array of overhead spotlights.

29. Apparatus as set forth in claim 28, wherein said operating room includes at least one other source of illumination and wherein said pointing controller comprises a microcontroller programmed to control said at least one other source of illumination and said array of spotlights in a coordinated manner.

30. Apparatus as set forth in claim 28,
wherein said operating room includes at least one further source of illumination,
wherein said apparatus further including sensors for detecting the state of the operating room, as such state changes from time to time during the course of a medical procedure being performed in the operating room, and
wherein said pointing controller comprises a microcontroller responsive to said sensors,
programmed with a set of rules defining the manner in which said at least one other source of illumination and said array of spotlights are to be controlled in a cooperative, coordinated manner in accordance with the state of the operating room, and
operative to control said other sources of illumination and said array of spotlights in accordance with said detected state, as such state changes from time to time, and said set of rules,
whereby said at least one other source of illumination and said array of spotlights are controlled in a cooperative, coordinated manner in accordance with the changing state of the operating room.

31. Apparatus as set forth in claim 28, wherein said at least one input device comprises an imager for viewing at least said region of surgical interest and providing an image signal representative thereof, and an image processor for processing said image signal.

32. Apparatus as set forth in claim 31, wherein said image processor comprises a microcontroller programmed to recognize and interpret gestures by individuals within the field of view of said imager, and for determining said portion of said region of surgical interest from said gestures.
